(19) United States
(12) Patent Application Publication
(10) Pub. No.: US 2024/0108704 A1
(43) Pub. Date: Apr. 4, 2024

(54) COMPOSITIONS, SYSTEMS, AND METHODS FOR TREATING CANCER USING ALTERNATING ELECTRIC FIELDS AND APOPTOTIC CANCER CELL VACCINATION

(71) Applicant: Novocure GmbH, Root (CH)

(72) Inventors: Tali Voloshin-Sela, Haifa (IL); Yiftah Barsheshet, Haifa (IL); Gadi Cohen, Haifa (IL)

(73) Assignee: Novocure GmbH, Root (CH)

(21) Appl. No.: 18/476,573

(22) Filed: Sep. 28, 2023

Related U.S. Application Data
(60) Provisional application No. 63/377,951, filed on Sep. 30, 2022.
(60) Provisional application No. 63/496,831, filed on Apr. 18, 2023.

(51) Int. Cl. A61K 39/00; A61K 41/10; A61P 35/00

(52) U.S. Cl. CPC A61K 39/0011; A61K 41/10; A61P 35/00; A61K 2039/5152; A61K 2039/55511

(57) ABSTRACT

Immunogenic compositions and systems are disclosed, as well as methods for producing the immunogenic compositions. Compositions, systems, and methods of reducing viability of cancer cells and treating cancer, as well as methods of reducing volume of a tumor and/or preventing an increase in volume of a tumor present in a body of a living subject, are also disclosed. The systems and methods involve application of an alternating electric field to cancer cell(s) (before or after isolation from a targeted portion of a subject), and irradiation of the isolated and treated cancer cell(s). The methods may further include administration of the irradiated, treated cancer cells back to the subject from which the cancer cell(s) were isolated, and/or application of an alternating electric field to the subject. The immunogenic compositions comprise populations of isolated, apoptotic cancer cells.

### CROSS-REFERENCE TO RELATED APPLICATIONS/INCORPORATION BY REFERENCE STATEMENT

The subject application claims benefit under 35 USC § 119(e) of US Provisional Application No. 63/377,951, filed Sep. 30, 2022; and US Provisional Application No. 63/496,831, filed Apr. 18, 2023. The entire contents of the above-referenced patent applications are hereby expressly incorporated herein by reference.

### BACKGROUND

Tumor Treating Fields (TTFields) are low intensity (e.g., 1-3 V/cm) alternating electric fields within the intermediate frequency range (such as, but not limited to, 100-500 kHz) that target solid tumors by disrupting mitosis. This non-invasive treatment targets solid tumors and is described, for example, in U.S. Pat. Nos. 7,016,725; 7,089,054; 7,333,852; 7,565,205; 8,244,345; 8,715,203; 8,764,675; 10,188,851; and 10,441,776. TTFields (or similar alternating electric fields utilized to treat other conditions) are typically delivered through two pairs of transducer arrays that generate perpendicular fields within the treated tumor; the electrode arrays that make up each of these pairs are positioned on opposite sides of the body part that is being treated. More specifically, for the OPTUNE® system, one pair of electrodes is located to the left and right (LR) of the tumor, and the other pair of electrodes is located anterior and posterior (AP) to the tumor. TTFields are approved for the treatment of glioblastoma multiforme (GBM), and may be delivered, for example, via the OPTUNE® system (Novocure Limited, St. Helier, Jersey), which includes transducer arrays placed on the patient's shaved head.

Each transducer array used for the delivery of TTFields in the OPTUNE® device comprises a set of ceramic disk electrodes, which are coupled to the patient's skin (such as, but not limited to, the patient's shaved head for treatment of GBM) through a layer of conductive medical gel. The purpose of the medical gel is to deform to match the body's contours and to provide good electrical contact between the arrays and the skin; as such, the gel interface bridges the skin and reduces interference. The device is intended to be continuously worn by the patient for 2-4 days before removal for hygienic care and re-shaving (if necessary), followed by reapplication with a new set of arrays. As such, the medical gel remains in substantially continuous contact with an area of the patient's skin for a period of 2-4 days at a time, and there is only a brief period of time in which the area of skin is uncovered and exposed to the environment before more medical gel is applied thereto.

Another cancer treatment modality involves cancer immunotherapy. The primary goal of cancer immunotherapy is to activate a preexisting, endogenous immune response in cancer patients. Certain possible targets of cancer immunotherapy include mutation-derived tumor specific antigens, or neoantigens, which are absent from normal cells and can be recognized by the immune system, thereby providing a specific target for antitumor therapy. Although significant advances have been made in the field, treatment efficacy still needs to be improved.

### DETAILED DESCRIPTION

Before explaining at least one embodiment of the inventive concept(s) in detail by way of exemplary language and results, it is to be understood that the inventive concept(s) is not limited in its application to the details of construction and the arrangement of the components set forth in the following description. The inventive concept(s) is capable of other embodiments or of being practiced or carried out in various ways. As such, the language used herein is intended to be given the broadest possible scope and meaning; and the embodiments are meant to be exemplary—not exhaustive. Also, it is to be understood that the phraseology and terminology employed herein is for the purpose of description and should not be regarded as limiting.

Unless otherwise defined herein, scientific and technical terms used in connection with the presently disclosed inventive concept(s) shall have the meanings that are commonly understood by those of ordinary skill in the art. Further, unless otherwise required by context, singular terms shall include pluralities and plural terms shall include the singular. The foregoing techniques and procedures are generally performed according to conventional methods well known in the art and as described in various general and more specific references that are cited and discussed throughout the present specification. The nomenclatures utilized in connection with, and the laboratory procedures and techniques of, analytical chemistry, synthetic organic chemistry, and medicinal and pharmaceutical chemistry described herein are those well-known and commonly used in the art. Standard techniques are used for chemical syntheses and chemical analyses.

All patents, published patent applications, and non-patent publications mentioned in the specification are indicative of the level of skill of those skilled in the art to which this presently disclosed inventive concept(s) pertains. All patents, published patent applications, and non-patent publications referenced in any portion of this application are herein expressly incorporated by reference in their entirety to the same extent as if each individual patent or publication was specifically and individually indicated to be incorporated by reference.

All of the compositions, assemblies, systems, kits, and/or methods disclosed herein can be made and executed without undue experimentation in light of the present disclosure. While the compositions, assemblies, systems, kits, and methods of the inventive concept(s) have been described in terms of particular embodiments, it will be apparent to those of skill in the art that variations may be applied to the compositions and/or methods and in the steps or in the sequence of steps of the methods described herein without departing from the concept, spirit, and scope of the inventive concept(s). All such similar substitutions and modifications apparent to those skilled in the art are deemed to be within the spirit, scope, and concept of the inventive concept(s) as defined by the appended claims.

As utilized in accordance with the present disclosure, the following terms, unless otherwise indicated, shall be understood to have the following meanings:

The use of the term “a” or “an” when used in conjunction with the term “comprising” in the claims and/or the specification may mean “one,” but it is also consistent with the meaning of “one or more,” “at least one,” and “one or more than one.” As such, the terms “a,” “an,” and “the” include plural referents unless the context clearly indicates otherwise. Thus, for example, reference to “a compound” may refer to one or more compounds, two or more compounds, three or more compounds, four or more compounds, or greater numbers of compounds. The term “plurality” refers to “two or more.”

The use of the term “at least one” will be understood to include one as well as any quantity more than one, including but not limited to, 2, 3, 4, 5, 10, 15, 20, 30, 40, 50, 100, etc. The term “at least one” may extend up to 100 or 1000 or more, depending on the term to which it is attached; in addition, the quantities of 100/1000 are not to be considered limiting, as higher limits may also produce satisfactory results. In addition, the use of the term “at least one of X, Y, and Z” will be understood to include X alone, Y alone, and Z alone, as well as any combination of X, Y, and Z. The use of ordinal number terminology (i.e., “first,” “second,” “third,” “fourth,” etc.) is solely for the purpose of differentiating between two or more items and is not meant to imply any sequence or order or importance to one item over another or any order of addition, for example.

The use of the term “or” in the claims is used to mean an inclusive “and/or” unless explicitly indicated to refer to alternatives only or unless the alternatives are mutually exclusive. For example, a condition “A or B” is satisfied by any of the following: A is true (or present) and B is false (or not present), A is false (or not present) and B is true (or present), and both A and B are true (or present).

As used herein, any reference to “one embodiment,” “an embodiment,” “some embodiments,” “one example,” “for example,” or “an example” means that a particular element, feature, structure, or characteristic described in connection with the embodiment is included in at least one embodiment. The appearance of the phrase “in some embodiments” or “one example” in various places in the specification is not necessarily all referring to the same embodiment, for example. Further, all references to one or more embodiments or examples are to be construed as non-limiting to the claims.

Throughout this application, the term “about” is used to indicate that a value includes the inherent variation of error for a composition/apparatus/device, the method being employed to determine the value, or the variation that exists among the study subjects. For example, but not by way of limitation, when the term “about” is utilized, the designated value may vary by plus or minus twenty percent, or fifteen percent, or twelve percent, or eleven percent, or ten percent, or nine percent, or eight percent, or seven percent, or six percent, or five percent, or four percent, or three percent, or two percent, or one percent from the specified value, as such variations are appropriate to perform the disclosed methods and as understood by persons having ordinary skill in the art.

As used in this specification and claim(s), the words “comprising” (and any form of comprising, such as “comprise” and “comprises”), “having” (and any form of having, such as “have” and “has”), “including” (and any form of including, such as “includes” and “include”), or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AAB, BBC, AAABCCCC, CBBAAA, CABABB, and so forth. The skilled artisan will understand that typically there is no limit on the number of items or terms in any combination, unless otherwise apparent from the context.

As used herein, the term “substantially” means that the subsequently described event or circumstance completely occurs or that the subsequently described event or circumstance occurs to a great extent or degree. For example, when associated with a particular event or circumstance, the term “substantially” means that the subsequently described event or circumstance occurs at least 80% of the time, or at least 85% of the time, or at least 90% of the time, or at least 95% of the time. For example, the term “substantially adjacent” may mean that two items are 100% adjacent to one another, or that the two items are within close proximity to one another but not 100% adjacent to one another, or that a portion of one of the two items is not 100% adjacent to the other item but is within close proximity to the other item.

The term “pharmaceutically acceptable” refers to compounds and compositions which are suitable for administration to humans and/or animals without undue adverse side effects such as (but not limited to) toxicity, irritation, and/or allergic response commensurate with a reasonable benefit/risk ratio.

The term “patient” or “subject” as used herein includes human and veterinary subjects. “Mammal” for purposes of treatment refers to any animal classified as a mammal, including (but not limited to) humans, domestic and farm animals, nonhuman primates, and any other animal that has mammary tissue.

The term “treatment” refers to both therapeutic treatment and prophylactic or preventative measures. Those in need of treatment include, but are not limited to, individuals already having a particular condition/disease/infection as well as individuals who are at risk of acquiring a particular condition/disease/infection (e.g., those needing prophylactic/preventative measures). The term “treating” refers to administering an agent/element/method to a patient for therapeutic and/or prophylactic/preventative purposes.

The term “therapeutic composition” or “pharmaceutical composition” as used herein refers to an agent that may be administered in vivo to bring about a therapeutic and/or prophylactic/preventative effect.

Administering a therapeutically effective amount or prophylactically effective amount is intended to provide a therapeutic benefit in the treatment, prevention, and/or management of a disease, condition, and/or infection. The specific amount that is therapeutically effective can be readily determined by the ordinary medical practitioner, and can vary depending on factors known in the art, such as (but not limited to) the type of condition/disease/infection, the patient's history and age, the stage of the condition/disease/infection, and the co-administration of other agents.

The term “effective amount” refers to an amount of a biologically active molecule or conjugate or derivative thereof, or an amount of a treatment protocol (e.g., an alternating electric field), sufficient to exhibit a detectable therapeutic effect without undue adverse side effects (such as (but not limited to) toxicity, irritation, and allergic response) commensurate with a reasonable benefit/risk ratio when used in the manner of the inventive concept(s). The therapeutic effect may include, for example but not by way of limitation, preventing, inhibiting, or reducing the occurrence of at least one condition, disease, and/or infection. The effective amount for a subject will depend upon the type of subject, the subject's size and health, the nature and severity of the condition/disease/infection to be treated, the method of administration, the duration of treatment, the nature of concurrent therapy (if any), the specific formulations employed, and the like. Thus, it is not possible to specify an exact effective amount in advance. However, the effective amount for a given situation can be determined by one of ordinary skill in the art using routine experimentation based on the information provided herein.

As used herein, the term “concurrent therapy” is used interchangeably with the terms “combination therapy” and “adjunct therapy,” and will be understood to mean that the patient in need of treatment is treated or given another drug for the condition/disease/infection in conjunction with the treatments of the present disclosure. This concurrent therapy can be sequential therapy, where the patient is treated first with one treatment protocol/pharmaceutical composition and then the other treatment protocol/pharmaceutical composition, or the two treatment protocols/pharmaceutical compositions are given simultaneously.

The terms “administration” and “administering,” as used herein, will be understood to include all routes of administration known in the art, including but not limited to, oral, topical, transdermal, parenteral, subcutaneous, intranasal, mucosal, intramuscular, intraperitoneal, intravitreal, and intravenous routes, and including both local and systemic applications. In addition, the compositions of the present disclosure (and/or the methods of administration of same) may be designed to provide delayed, controlled, or sustained release using formulation techniques which are well known in the art.

Turning now to the inventive concept(s), a combinatorial therapy for cancer is disclosed herein. The combinatorial therapy combines (i) production of alternating electric field-treated apoptotic cancer cells (such as, but not limited to, TTField-treated apoptotic cancer cells) via application of an alternating electric field (such as, but not limited to, a TTField) thereto; (ii) irradiation of the TTField-treated apoptotic cells; (iii) administration of the alternating electric field-treated apoptotic cancer cells to a subject; and (iv) application of an alternating electric field (such as, but not limited to, a TTField) to the target region of the subject. The combination of alternating electric field treatment with apoptotic cancer cell vaccination provides a synergistic result in the treatment of cancer.

Certain non-limiting embodiments of the present disclosure are directed to methods of treating cancer in a subject and/or methods of reducing a volume of a tumor and/or preventing an increase of volume of the tumor present in a subject. The methods include the steps of: (1) isolating cancer cells from a target region of the subject; (2) applying an alternating electric field to the isolated cancer cells; (3) irradiating the isolated cancer cells to which the alternating electric field has been applied; (4) administering the isolated, irradiated cancer cells to the subject; and (5) applying an alternating electric field to the target region of the subject.

Certain non-limiting embodiments of the present disclosure are directed to other similar methods of treating cancer in a subject (and/or methods of reducing a volume of a tumor and/or preventing an increase of volume of the tumor present in a subject), with the exception that the alternating electric field is applied to a target region of the subject prior to isolating cancer cells from the target region of the subject (such as, but not limited to, to induce an immunogenic response). In other words, the methods of the present disclosure cover application of alternating electric fields to the cancer cells both in vivo as well as ex vivo prior to irradiating the alternating electric field-treated cancer cells and administering said cells to the subject.

The methods of the present disclosure may be utilized to treat any types of cancer cells/cancers/tumors that respond to alternating electric field/TTFields and/or apoptotic cancer cell treatment. Non-limiting examples of cancer cells/cancers/tumors that can be treated in accordance with the present disclosure include hepatocellular carcinomas, glioblastomas, pleural mesotheliomas, differentiated thyroid cancers, advanced renal cell carcinomas, ovarian cancers, cervical cancers, breast cancers, pancreatic cancers, lung cancers (such as, but not limited to, non-small cell lung cancers), and the like, as well as any combination thereof.

Any type of conductive or non-conductive electrode(s) and/or transducer array(s) that can be utilized for generating an alternating electric field that are known in the art or otherwise contemplated herein may be utilized for generation of the alternating electric field in accordance with the methods of the present disclosure. Non-limiting examples of electrodes and transducer arrays that can be utilized for generating an alternating electric field in accordance with the present disclosure include those that function as part of an alternating electric field-generating system (e.g., TTFields system) as described, for example but not by way of limitation, in U.S. Pat. Nos. 7,016,725; 7,089,054; 7,333,852; 7,565,205; 8,244,345; 8,715,203; 8,764,675; 10,188,851; and 10,441,776; and in US Patent Application Nos. US 2018/0160933; US 2019/0117956; US 2019/0307781; and US 2019/0308016.

The alternating electric field may be generated at any frequency in accordance with the present disclosure. For example (but not by way of limitation), the alternating electric field may have a frequency of about 50 kHz, about 75 kHz, about 100 kHz, about 125 kHz, about 150 kHz, about 175 kHz, about 200 kHz, about 225 kHz, about 250 kHz, about 275 kHz, about 300 kHz, about 325 kHz, about 350 kHz, about 375 kHz, about 400 kHz, about 425 kHz, about 450 kHz, about 475 kHz, about 500 kHz, about 550 kHz, about 600 kHz, about 650 kHz, about 700 kHz, about 750 kHz, about 800 kHz, about 850 kHz, about 900 kHz, about 950 kHz, about 1 MHz, about 2 MHz, about 3 MHz, about 4 MHz, about 5 MHz, about 6 MHz, about 7 MHz, about 8 MHz, about 9 MHz, about 10 MHz, and the like, as well as a range formed from any of the above values (e.g., a range of from about 50 kHz to about 10 MHz, a range of from about 50 kHz to about 1 MHz, a range of from about 100 kHz to about 500 kHz, a range of from about 150 kHz to about 300 kHz, etc.), and a range that combines two integers that fall between two of the above-referenced values (e.g., a range of from about 122 kHz to about 313 kHz, a range of from about 78 kHz to about 298 kHz, etc.).

In certain particular (but non-limiting) embodiments, the alternating electric field may be imposed at two or more different frequencies. When two or more frequencies are present, each frequency is selected from any of the above-referenced values, or a range formed from any of the above-referenced values, or a range that combines two integers that fall between two of the above-referenced values.

The alternating electric field may have any field strength in the subject/cancer cells, so long as the alternating electric field is capable of functioning in accordance with the present disclosure. For example (but not by way of limitation), the alternating electric field may have a field strength of at least about 1 V/cm, about 1.5 V/cm, about 2 V/cm, about 2.5 V/cm, about 3 V/cm, about 3.5 V/cm, about 4 V/cm, about 4.5 V/cm, about 5 V/cm, about 5.5 V/cm, about 6 V/cm, about 6.5 V/cm, about 7 V/cm, about 7.5 V/cm, about 8 V/cm, about 9 V/cm, about 9.5 V/cm, about 10 V/cm, about 10.5 V/cm, about 11 V/cm, about 11.5 V/cm, about 12 V/cm, about 12.5 V/cm, about 13 V/cm, about 13.5 V/cm, about 14 V/cm, about 14.5 V/cm, about 15 V/cm, about 15.5 V/cm, about 16 V/cm, about 16.5 V/cm, about 17 V/cm, about 17.5 V/cm, about 18 V/cm, about 18.5 V/cm, about 19 V/cm, about 19.5 V/cm, about 20 V/cm, and the like, as well as a range formed from any of the above values (e.g., a range of from about 1 V/cm to about 20 V/cm, a range of from about 1 V/cm to about 10 V/cm, a range of from about 1 V/cm to about 4 V/cm, etc.), and a range that combines two integers that fall between two of the above-referenced values (e.g., a range of from about 1.1 V/cm to about 18.6 V/cm, a range of from about 1.2 V/cm to about 9.8 V/cm, a range of from about 1.3 V/cm to about 4.7 V/cm, etc.).

The alternating electric field may be applied for any period of time sufficient to achieve a desired result in accordance with the present disclosure (such as, but not limited to, generation of an immune response, alteration of cancer cells to induce an immunogenic response, reduction in viability of cancer cells, and/or a reduction in tumor volume (and/or a prevention of increase in tumor volume, and the like). For example, but not by way of limitation, the alternating electric field may be applied for at least about 1 minute, about 5 minutes, about 10 minutes, about 15 minutes, about 20 minutes, about 25 minutes, about 30 minutes, about 35 minutes, about 40 minutes, about 45 minutes, about 50 minutes, about 55 minutes, about 1 hour, about 2 hours, about 3 hours, about 4 hours, about 5 hours, about 6 hours, about 7 hours, about 8 hours, about 9 hours, about 10 hours, about 11 hours, about 12 hours, about 15 hours, about 18 hours, about 21 hours, about 24 hours, about 27 hours, about 30 hours, about 33 hours, about 36 hours, about 39 hours, about 42 hours, about 45 hours, about 48 hours, about 51 hours, about 54 hours, about 57 hours, about 60 hours, about 63 hours, about 66 hours, about 69 hours, about 72 hours, about 75 hours, about 78 hours, about 81 hours, about 84 hours, about 87 hours, about 90 hours, about 93 hours, about 96 hours, about 1 day, about 2 days, about 3 days, about 4 days, about 5 days, about 6 days, about 7 days, and the like, as well as a range formed from any of the above values (e.g., a range of from about 1 minute to about 12 hours, a range of from about 1 minute to about 1 hour, a range of from about 1 hour to about 7 days, a range of from about 24 hours to about 72 hours, etc.), and a range that combines two integers that fall between two of the above-referenced values (e.g., a range of from about 14 hours to about 68 hours, etc.).

In a particular (but non-limiting) embodiment, the alternating electric field is applied in vivo at a frequency in a range of from about 50 kHz to about 1 MHz, a field strength of at least about 1 V/cm (e.g., in a range from about 1 V/cm to about 10 V/cm), and a period of time that is at least about 24 hours.

In a particular (but non-limiting) embodiment, the alternating electric field is applied ex vivo at a frequency in a range of from about 50 kHz to about 1 MHz, a field strength of at least about 1 V/cm (e.g., in a range of from about 1 V/cm to about 10 V/cm), and a period of time that is in a range of from about 1 hour to about 7 days.

Any methods of irradiating cancer cells known in the art or otherwise contemplated herein may be utilized in accordance with the present disclosure, so long as the cancer cells are irradiated at a sufficient strength of radiation and for a sufficient amount of time to render the cancer cells non-viable/proliferation incompetent. For example, but not by way of limitation, the cancer cells may be irradiated at a strength of at least about 1 gray (Gy). The ability to select a sufficient radiation strength and a sufficient period of time is well within the purview of a person of ordinary skill in the art, and thus no further description thereof is deemed necessary.

The alternating electric field-treated cancer cells are irradiated for any period of time sufficient to achieve an apoptotic state and render the cancer cells substantially non-viable. For example, but not by way of limitation, the cells may be irradiated for at least about 30 minutes, about 1 hour, about 2 hours, about 3 hours, about 4 hours, about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, about 24 hours, and the like, as well as a range formed from any of the above values (e.g., a range of from about 30 minutes to about 24 hours, etc.), and a range that combines two integers that fall between two of the above-referenced values (e.g., a range of from about 45 minutes to about 20.5 hours, etc.).

In a particular (but non-limiting) embodiment, the period of time that the alternating electric field-treated cancer cells are irradiated is in a range of from about 24 hours to about 96 hours.

In certain particular (but non-limiting) embodiments, the non-viable, irradiated cancer cells are isolated prior to administration to the subject (i.e., between steps (3) and (4) of the methods described above). This optional isolation step ensures that the composition administered to the subject does not contain any cancer cells that may not have received enough radiation and are still viable. For example (but not by way of limitation), the cells may be exposed to one or more sorting steps to isolate non-viable cells and ensure that no live cells are present in the sorted cells.

Alternatively, the irradiation conditions may be stringent enough (i.e., via a long duration and/or high intensity) to ensure that no cells present in the irradiated sample are still viable. In this instance, the non-viable, irradiated cancer cells can be directly administered to the subject.

The alternating electric field-treated, irradiated cancer cells may be disposed and administered in any formulation known in the art or otherwise contemplated herein that will allow the apoptotic cancer cells to have a deleterious effect on the cancer present in the subject. For example, but not by way of limitation, the alternating electric field-treated, irradiated cancer cells may be administered in the form of a pharmaceutical composition that comprises the cells in combination with at least one pharmaceutically-acceptable carrier. Non-limiting examples of suitable pharmaceutically acceptable carriers that may be utilized in accordance with the present disclosure include water; saline; dextrose solutions; fructose or mannitol; calcium carbonate; cellulose; ethanol; oils of animal, vegetative, or synthetic origin; carbohydrates, such as glucose, sucrose, or dextrans; antioxidants, such as ascorbic acid or glutathione; chelating agents; low molecular weight proteins; detergents; liposomal carriers; buffered solutions, such as sodium chloride, saline, phosphate-buffered saline, and/or other substances which are physiologically acceptable and/or safe for use; diluents; excipients such as polyethylene glycol (PEG); or any combination thereof. Suitable pharmaceutically acceptable carriers for pharmaceutical formulations are described, for example, in Remington: The Science and Practice of Pharmacy, 23rd ed (2020).

In certain non-limiting embodiments, the pharmaceutical composition containing the alternating electric field-treated, irradiated cancer cells may further be formulated as an immunogenic composition. The immunogenic composition may contain the same components as the pharmaceutical composition described above (i.e., the apoptotic cancer cells plus the pharmaceutically-acceptable carrier). In certain particular (but non-limiting) embodiments, the immunogenic composition may further include at least one additional agent. Non-limiting examples of agents that may be included as part of the immunogenic composition include an adjuvant, a cytokine, an interferon, a TLR agonist, a STING (stimulator of interferon genes) agonist, GM-CSF, CD40L, Fms related tyrosine kinase 3 ligand (FLT3L), a C type Lectin Receptor (CLR), an anti-LAG3 agent (such as, but not limited to, OPDUALAG™ and/or Relatimab (Bristol-Myers Squibb, New York, NY)), other active agents, and the like, as well as any combinations thereof.

In addition, any of the apoptotic cancer cell-containing compositions of the present disclosure may contain other agents that allow for administration of the compositions via a particular administration route. For example, but not by way of limitation, the compositions may be formulated for administration by oral, topical, transdermal, intradermal, parenteral, subcutaneous, intranasal, mucosal, intramuscular, intraperitoneal, intravitreal, intravenous, and/or intranodal routes. Based on the route of administration, the compositions may also contain one or more additional components in addition to the active agent (e.g., apoptotic cancer cells, immunogenic composition, and/or additional therapeutic agent(s)). Examples of additional secondary compounds that may be present include, but are not limited to, fillers, salts, buffers, preservatives, stabilizers, solubilizers, wetting agents, emulsifying agents, dispersing agents, and other materials well known in the art.

In a particular (but non-limiting) embodiment, the composition containing the apoptotic cancer cells is administered intradermally, subcutaneously, intravenously, and/or intranodally to the subject.

In certain non-limiting embodiments, the method may further include an additional step of applying the alternating electric field to the target region of the subject following isolation of the cancer cells (and/or resection of the tumor) and prior to or following administration of the apoptotic cancer cell-containing composition. When this additional alternating electric field application step is present, the alternating electric field may be applied simultaneously or wholly or partially sequentially with the administration of the apoptotic cancer cell-containing composition. In certain particular (but non-limiting) embodiments, the alternating electric field may be applied after the apoptotic cancer cell-containing composition is administered. In other particular (but non-limiting) embodiments, the alternating electric field may be applied at the same time or after administration of the apoptotic cancer cell-containing composition. In another particular (but non-limiting) embodiment, the apoptotic cancer cell-containing composition may be administered during application of the alternating electric field (i.e., before the period of time that the alternating electric field is applied has elapsed).

For example (but not by way of limitation), the apoptotic cancer cell-containing composition may be administered before the additional application of the alternating electric field has commenced by a period of at least about 1 minute, about 5 minutes, about 10 minutes, about 15 minutes, about 30 minutes, about 45 minutes, about 1 hour, about 1.5 hours, about 2 hours, about 3 hours, about 4 hours, about 5 hours, about 6 hours, about 7 hours, about 8 hours, about 9 hours, about 10 hours, about 12 hours, about 15 hours, about 18 hours, about 21 hours, about 24 hours, about 27 hours, about 30 hours, about 33 hours, about 36 hours, about 39 hours, about 42 hours, about 45 hours, about 48 hours, about 51 hours, about 54 hours, about 57 hours, about 60 hours, about 63 hours, about 66 hours, about 69 hours, about 72 hours, about 75 hours, about 78 hours, about 81 hours, about 84 hours, about 87 hours, about 90 hours, about 93 hours, about 96 hours, and the like, as well as a range formed from any of the above values (e.g., a range of from about 1 minute to about 24 hours, etc.), and a range that combines two integers that fall between two of the above-referenced values (e.g., a range of from about 14 minutes to about 94 hours, etc.).

In other non-limiting examples, the apoptotic cancer cell-containing composition may be administered after the additional application of the alternating electric field has commenced by a period of at least about 1 minute, about 5 minutes, about 10 minutes, about 15 minutes, about 30 minutes, about 45 minutes, about 1 hour, about 1.5 hours, about 2 hours, about 3 hours, about 4 hours, about 5 hours, about 6 hours, about 7 hours, about 8 hours, about 9 hours, about 10 hours, about 12 hours, about 15 hours, about 18 hours, about 21 hours, about 24 hours, about 27 hours, about 30 hours, about 33 hours, about 36 hours, about 39 hours, about 42 hours, about 45 hours, about 48 hours, about 51 hours, about 54 hours, about 57 hours, about 60 hours, about 63 hours, about 66 hours, about 69 hours, about 72 hours, about 75 hours, about 78 hours, about 81 hours, about 84 hours, about 87 hours, about 90 hours, about 93 hours, about 96 hours, and the like, as well as a range formed from any of the above values (e.g., a range of from about 1 minute to about 24 hours, etc.), and a range that combines two integers that fall between two of the above-referenced values (e.g., a range of from about 14 minutes to about 94 hours, etc.).

In yet other non-limiting examples, the apoptotic cancer cell-containing composition may be administered after the period that the additional alternating electric field is applied has elapsed, wherein the apoptotic cancer cell-containing composition is administered within about 1 minute, about 5 minutes, about 10 minutes, about 15 minutes, about 30 minutes, about 45 minutes, about 1 hour, about 1.5 hours, about 2 hours, about 3 hours, about 4 hours, about 5 hours, about 6 hours, about 7 hours, about 8 hours, about 9 hours, about 10 hours, about 12 hours, about 15 hours, about 18 hours, about 21 hours, about 24 hours, about 27 hours, about 30 hours, about 33 hours, about 36 hours, about 39 hours, about 42 hours, about 45 hours, about 48 hours, about 51 hours, about 54 hours, about 57 hours, about 60 hours, about 63 hours, about 66 hours, about 69 hours, about 72 hours, about 75 hours, about 78 hours, about 81 hours, about 84 hours, about 87 hours, about 90 hours, about 93 hours, about 96 hours, and the like, of when the period of time elapsed.

The apoptotic cancer cell-containing composition(s) may be administered to the subject at any concentration that is capable of inducing an inflammatory response to the tumor or cancer cells. For example, but not by way of limitation, the apoptotic cancer cells may be administered at about 10 cells/kg body weight, about 100 cells/kg body weight, about 1000 cells/kg body weight, about 10⁴ cells/kg body weight, about 10⁵ cells/kg body weight, about 10⁶ cells/kg body weight, about 10⁷ cells/kg body weight, about 10⁸ cells/kg body weight, about 10⁹ cells/kg body weight, about 10¹⁰ cells/kg body weight, about 10¹¹ cells/kg body weight, about 10¹² cells/kg body weight, about 10¹³ cells/kg body weight, about 10¹⁴ cells/kg body weight, about 10¹⁵ cells/kg body weight, or higher, as well as a range formed from any of the above values (e.g., a range of from about 10⁴ to about 10⁹ cells/kg body weight, etc.).

In certain particular (but non-limiting) embodiments, the method further involves concurrent therapy with two or more compositions. As such, the method may include an additional step of administering at least a second composition to the subject. Additional non-limiting examples of therapeutic agents that can be utilized as part of a second composition administered simultaneously or wholly or partially sequentially with the apoptotic cancer cell-containing composition include Lenvatinib, Pembrolizumab, and other anti-PD-1 therapeutics such as (but not limited to) Tislelizumab, Nivolumab, and Cemiplimab; an anti-LAG3 agent such as (but not limited to) OPDUALAG™ and/or Relatimab (Bristol-Myers Squibb, New York, NY); an anti-PD-L1 therapeutic agent, such as (but not limited to) Atezolizumab, Avelumab, and Durvalumab; an anti-CTLA-4 therapeutic agent, such as (but not limited to) Ipilimumab; chemotherapeutic agents, such as (but not limited to) Paclitaxel, Docetaxel, Ifosamide, Etoposide (Vepesid), Gemcitabine, Lomustine, Nab Paclitaxel, Temozolomide, and Carboplatin; TKI inhibitors, such as (but not limited to) Everolimus; mTOR inhibitors; Akt inhibitors; PI3K inhibitors; PARP inhibitors; VEGF inhibitors; FGF inhibitors; aromatase inhibitors (such as (but not limited to) Letrozole); biologics such as monoclonal antibodies (such as, but not limited to, Denosumab and Pembrolizumab); and the like, as well as any combinations thereof.

When present, the concurrent therapy may be performed substantially simultaneously or wholly or partially sequentially with the administration of the apoptotic cancer cell-containing composition. In addition, the two compositions may be administered via the same route (e.g., both orally administered or injected), or the two compositions may be administered by different routes (e.g., one composition orally administered and another composition intravenously administered).

When both the steps of administering a second composition and applying an alternating electric field to the subject to which the apoptotic cancer cell-containing composition has been administered are present, the optional administration step may be performed before or after the application of the alternating electric field has begun, and during application of the alternating electric field and/or after application of the alternating electric field has elapsed, in the same manner(s) and time frame(s) as described above for the apoptotic cancer cell-containing composition.

That is, for example (but not by way of limitation), the second composition may be administered after application of the alternating electric field has commenced by a period of at least about 3 hours, about 6 hours, about 9 hours, about 12 hours, about 15 hours, about 18 hours, about 21 hours, about 24 hours, about 27 hours, about 30 hours, about 33 hours, about 36 hours, about 39 hours, about 42 hours, about 45 hours, about 48 hours, about 51 hours, about 54 hours, about 57 hours, about 60 hours, about 63 hours, about 66 hours, about 69 hours, about 72 hours, about 75 hours, about 78 hours, about 81 hours, about 84 hours, about 87 hours, about 90 hours, about 93 hours, about 96 hours, and the like, as well as a range formed from any of the above values (e.g., a range of from about 24 hours to about 96 hours, etc.), and a range that combines two integers that fall between two of the above-referenced values (e.g., a range of from about 14 hours to about 94 hours, etc.). In a particular (but non-limiting) embodiment, the second composition is administered at least about 24 hours after application of the alternating electric field has begun.

In other non-limiting examples, the second composition may be administered after the period of time that the alternating electric field is applied has elapsed, wherein the second composition is administered within about 3 hours, about 6 hours, about 9 hours, about 12 hours, about 15 hours, about 18 hours, about 21 hours, about 24 hours, about 27 hours, about 30 hours, about 33 hours, about 36 hours, about 39 hours, about 42 hours, about 45 hours, about 48 hours, about 51 hours, about 54 hours, about 57 hours, about 60 hours, about 63 hours, about 66 hours, about 69 hours, about 72 hours, about 75 hours, about 78 hours, about 81 hours, about 84 hours, about 87 hours, about 90 hours, about 93 hours, about 96 hours, and the like, of when the period of time elapsed. In a particular (but non-limiting) embodiment, the second composition is administered within about 96 hours of when the period of time elapsed.

In addition, for example (but not by way of limitation), the second composition may be administered after administration of the apoptotic cancer cell-containing composition by a period of at least about 1 minute, about 5 minutes, about 10 minutes, about 15 minutes, about 30 minutes, about 45 minutes, about 1 hour, about 2 hours, about 3 hours, about 4 hours, about 5 hours, about 6 hours, about 7 hours, about 8 hours, about 9 hours, about 10 hours, about 11 hours, about 12 hours, about 15 hours, about 18 hours, about 21 hours, about 24 hours, about 27 hours, about 30 hours, about 33 hours, about 36 hours, about 39 hours, about 42 hours, about 45 hours, about 48 hours, about 51 hours, about 54 hours, about 57 hours, about 60 hours, about 63 hours, about 66 hours, about 69 hours, about 72 hours, about 75 hours, about 78 hours, about 81 hours, about 84 hours, about 87 hours, about 90 hours, about 93 hours, about 96 hours, and the like, as well as a range formed from any of the above values (e.g., a range of from about 24 hours to about 96 hours, etc.), and a range that combines two integers that fall between two of the above-referenced values (e.g., a range of from about 14 hours to about 94 hours, etc.). In a particular (but non-limiting) embodiment, the second composition is administered at least about 12 hours after administration of the apoptotic cancer cell-containing composition.

In certain particular (but non-limiting) embodiments, the method may further comprise the step of administering at least one additional therapy to the subject. Any therapies known in the art or otherwise contemplated herein for use with alternating electric fields and/or apoptotic cancer cell therapy may be utilized in accordance with the methods of the present disclosure. Non-limiting examples of additional therapies that may be utilized include radiation therapy, photodynamic therapy, transarterial chemoembolization (TACE), or combinations thereof.

In certain particular (but non-limiting) embodiments, the method includes one or more additional steps. For example (but not by way of limitation), the method may further include repeating any of the steps one or more times. Each of the steps can be repeated as many times as necessary. When application of the alternating electric field is repeated, the transducer arrays may be placed in slightly different positions on the subject than their original placement; relocation of the arrays in this manner may further aid in treatment of the tumor/cancer. In addition, any of the administration steps (including the step of administering the apoptotic cancer cell-containing composition as well as any optional administration steps) may be repeated various times and at various intervals to follow any known and/or generally accepted dosage/treatment regimen for the composition(s)/therapy(ies).

Certain non-limiting embodiments of the present disclosure are related to immunogenic compositions that comprise a population of any of the isolated, irradiated, apoptotic cancer cells produced as described or otherwise contemplated herein. In certain particular (but non-limiting) embodiments, the apoptotic cancer cells are produced by exposing the isolated cancer cells to an alternating electric field (either in vivo or ex vivo) and then irradiating the cancer cells to which the alternating electric field has been applied.

The immunogenic composition may be formulated for administration by any of the administration routes disclosed or otherwise contemplated herein. In certain particular (but non-limiting) embodiments, the immunogenic composition is formulated for intradermal, subcutaneous, intravenous, and/or intranodal administration.

In certain particular (but non-limiting) embodiments, the immunogenic composition may further include one or more additional active agents that further assists in stimulating the immune system to recognize and attack the cancer cells in the subject. Non-limiting examples of additional agents that may be present in the immunogenic composition include an adjuvant, a cytokine, an interferon, a TLR agonist, a STING (stimulator of interferon genes) agonist, GM-CSF, CD40L, Fms related tyrosine kinase 3 ligand (FLT3L), a C type Lectin Receptor (CLR), an anti-LAG3 agent (such as, but not limited to, OPDUALAG™ and/or Relatimab (Bristol-Myers Squibb, New York, NY)), and combinations thereof.

Certain non-limiting embodiments of the present disclosure are related to kits that include any of the components of the alternating electric field-generating systems (such as, but not limited to, one or more transducer arrays and/or one or more hydrogel compositions, as disclosed in U.S. Pat. Nos. 7,016,725; 7,089,054; 7,333,852; 7,565,205; 8,244,345; 8,715,203; 8,764,675; 10,188,851; and 10,441,776; and in US Patent Application Nos. US 2018/0160933; US 2019/0117956; US 2019/0307781; and US 2019/0308016) in combination with one or more component, device, and/or reagent utilized in one or more of the method steps, including the steps of isolating the cancer cells, irradiating the cancer cells, isolating the irradiated, treated apoptotic cancer cells, and/or formulating said isolated apoptotic cancer cells for administration to the subject, in accordance with the methods disclosed or otherwise contemplated herein. The kits may optionally further include one or more of any of the optional compositions disclosed or otherwise contemplated herein (such as, but not limited to, one or more compositions utilized in an optional concurrent therapy step(s)). The kits may optionally further include one or more devices (or one or more components of devices) utilized in one or more additional therapy steps.

In a particular (but non-limiting) embodiment, the kit may further include instructions for performing any of the methods disclosed or otherwise contemplated herein. For example (but not by way of limitation), the kit may include instructions for applying one or more components of the alternating electric field-generating system to the skin of the patient, instructions for applying the alternating electric field to the patient, instructions for when and how to administer the apoptotic cancer cell-containing composition(s) and optionally how to administer one or more optional additional compositions, and/or instructions for when to activate and turn off the alternating electric field in relation to the administration of the apoptotic cancer cell-containing composition(s) and/or administration of one or more optional compositions.

In addition to the components described in detail herein above, the kits may further contain other component(s)/reagent(s) for performing any of the particular methods described or otherwise contemplated herein. For example (but not by way of limitation), the kits may additionally include: (i) components for preparing the skin prior to disposal of the hydrogel compositions and/or transducer arrays thereon (i.e., a razor, a cleansing composition or wipe/towel, etc.); (ii) components for removal of the gel/transducer array(s); (iii) components for cleansing of the skin after removal of the gel/transducer array(s); (iv) components for isolation of the cancer cells/portion of tumor; (v) components for irradiation of the isolated, alternating electric field-treated cancer cells/portion of tumor; and/or (vi) components for isolation of the irradiated, alternating electric field-treated cancer cells. The nature of these additional component(s)/reagent(s) will depend upon the particular treatment format, and identification thereof is well within the skill of one of ordinary skill in the art; therefore, no further description thereof is deemed necessary. Also, the components/reagents present in the kits may each be in separate containers/compartments, or various components/reagents can be combined in one or more containers/compartments, depending on the sterility, cross-reactivity, and stability of the components/reagents.

The kit may be disposed in any packaging that allows the components present therein to function in accordance with the present disclosure. In certain non-limiting embodiments, the kit further comprises a sealed packaging in which the components are disposed. In certain particular (but non-limiting) embodiments, the sealed packaging is substantially impermeable to air and/or substantially impermeable to light.

In addition, the kit can further include a set of written instructions explaining how to use one or more components of the kit. A kit of this nature can be used in any of the methods described or otherwise contemplated herein.

In certain non-limiting embodiments, the kit has a shelf life of at least about six months, such as (but not limited to), at least about nine months, or at least about 12 months.

Certain non-limiting embodiments of the present disclosure are related to systems that include any of the components of the alternating electric field-generating systems (such as, but not limited to, one or more transducer arrays and/or one or more hydrogel compositions, as disclosed in U.S. Pat. Nos. 7,016,725; 7,089,054; 7,333,852; 7,565,205; 8,244,345; 8,715,203; 8,764,675; 10,188,851; and 10,441,776; and in US Patent Application Nos. US 2018/0160933; US 2019/0117956; US 2019/0307781; and US 2019/0308016) in combination with at least one of any of the compositions comprising apoptotic cancer cells produced as disclosed or otherwise contemplated herein. The systems may optionally further include one or more of any of the optional compositions disclosed or otherwise contemplated herein. The systems may optionally further include one or more devices (or one or more components of devices) utilized in the various isolation, treatment, irradiation, or administration steps, or optional additional treatment/therapy steps.

### EXAMPLE

An Example is provided herein below. However, the present disclosure is to be understood to not be limited in its application to the specific experimentation, results, and laboratory procedures disclosed herein after. Rather, the Example is simply provided as one of various embodiments and is meant to be exemplary, not exhaustive.

The primary goal of cancer immunotherapy is to activate a preexisting, endogenous immune response in cancer patients. Although significant advances have been made in this field, treatment efficacy still needs to be improved. A personalized cancer vaccine is a promising strategy to strengthen the anti-tumor immune response via an immunogenic form of apoptosis, also known as immunogenic cell death (ICD). ICD is characterized by the emission of danger-associated molecular patterns that serve to recruit immune cells to the site of the tumor. Previous studies have shown that TTFields treatment potentiates immunogenic cell death in cancer cells, ultimately stimulating an immune response through engulfing cancer cells. In turn, it presents neoantigens to initiate adaptive immunity further.

To support the rationale for using TTFields as an immune modulator, mice are treated with TTFields for 72 h using the INOVITRO™ system (Novocure GmbH, Root, Switzerland). Cancer cells are then isolated from the mice and irradiated. The irradiated, apoptotic cancer cells are isolated away from the culture, and the apoptotic cancer cells are administered to mice as a vaccine to trigger an immune response to cancer development.

In this manner, methods of increasing immunity to cancer cells are combined with TTFields treatment. The combination of TTFields treatment with administration of a personalized apoptotic cancer cell-containing composition provides a synergistic effect over either treatment alone and initiates an immune response in the patient that will allow the immune system to eliminate the cancer cells.

Non-Limiting Illustrative Embodiments of the Inventive Concept(s)

Illustrative embodiment 1. A method of preparing an immunogenic composition, the method comprising the steps of: (1) isolating cancer cells from a target region of the subject; (2) applying an alternating electric field to the isolated cancer cells; and (3) irradiating the isolated cancer cells to which the alternating electric field has been applied to form the immunogenic composition.

Illustrative embodiment 1a. A method of preparing an immunogenic composition, the method comprising the steps of: (a) applying an alternating electric field to cancer cells isolated from a target region of a subject; and (b) irradiating the isolated cancer cells to which the alternating electric field has been applied to form the immunogenic composition.

Illustrative embodiment 2. A method of preparing an immunogenic composition, the method comprising the steps of: (i) applying an alternating electric field to a target region of the subject; (2) isolating cancer cells to which the alternating electric field has been applied from the target region of the subject; (3) irradiating the isolated cancer cells to which the alternating electric field has been applied to form the immunogenic composition.

Illustrative embodiment 2a. A method of preparing an immunogenic composition, the method comprising the steps of: (a) isolating cancer cells from a target region of a subject, wherein an alternating electric field has been applied to the target region of the subject prior to isolation of the cancer cells; and (b) irradiating the isolated cancer cells to which the alternating electric field has been applied to form the immunogenic composition.

Illustrative embodiment 3. A method of treating cancer in a subject, the method comprising the steps of: (1) isolating cancer cells from a target region of the subject; (2) applying an alternating electric field to the isolated cancer cells; (3) irradiating the isolated cancer cells to which the alternating electric field has been applied; (4) administering the isolated, irradiated cancer cells to the subject; and (5) applying an alternating electric field to the target region of the subject.

Illustrative embodiment 3a. A method of treating cancer in a subject, the method comprising the steps of: (a) applying an alternating electric field to cancer cells isolated from a target region of the subject; (b) irradiating the isolated cancer cells to which the alternating electric field has been applied; (c) administering the isolated, irradiated cancer cells to the subject; and (d) applying an alternating electric field to the target region of the subject.

Illustrative embodiment 4. A method of treating cancer in a subject, the method comprising the steps of: (i) applying an alternating electric field to a target region of the subject; (2) isolating cancer cells to which the alternating electric field has been applied from the target region of the subject; (3) irradiating the isolated cancer cells to which the alternating electric field has been applied; (4) administering the isolated, irradiated cancer cells to the subject; and (5) applying an alternating electric field to the target region of the subject.

Illustrative embodiment 4a. A method of treating cancer in a subject, the method comprising the steps of: (a) isolating cancer cells from a target region of the subject, wherein an alternating electric filed has been applied the target region of the subject prior to isolation of the cancer cells; (b) irradiating the isolated cancer cells to which the alternating electric field has been applied; (c) administering the isolated, irradiated cancer cells to the subject; and (d) applying an alternating electric field to the target region of the subject.

Illustrative embodiment 5. A method of reducing a volume of a tumor and/or preventing an increase of volume of the tumor, wherein the tumor is present in a body of a living subject and includes a plurality of cancer cells, the method comprising the steps of: (1) isolating cancer cells from a target region of the subject; (2) applying an alternating electric field to the isolated cancer cells; (3) irradiating the isolated cancer cells to which the alternating electric field has been applied; (4) administering the isolated, irradiated cancer cells to the subject; and (5) applying an alternating electric field to the target region of the subject.

Illustrative embodiment 5a. A method of reducing a volume of a tumor and/or preventing an increase of volume of the tumor, wherein the tumor is present in a body of a living subject and includes a plurality of cancer cells, the method comprising the steps of: (a) applying an alternating electric field to cancer cells isolated from a target region of the subject; (b) irradiating the isolated cancer cells to which the alternating electric field has been applied; (c) administering the isolated, irradiated cancer cells to the subject; and (d) applying an alternating electric field to the target region of the subject.

Illustrative embodiment 6. A method of reducing a volume of a tumor and/or preventing an increase of volume of the tumor, wherein the tumor is present in a body of a living subject and includes a plurality of cancer cells, the method comprising the steps of: (i) applying an alternating electric field to a target region of the subject; (2) isolating cancer cells to which the alternating electric field has been applied from the target region of the subject; (3) irradiating the isolated cancer cells to which the alternating electric field has been applied; (4) administering the isolated, irradiated cancer cells to the subject; and (5) applying an alternating electric field to the target region of the subject.

Illustrative embodiment 6a. A method of reducing a volume of a tumor and/or preventing an increase of volume of the tumor, wherein the tumor is present in a body of a living subject and includes a plurality of cancer cells, the method comprising the steps of: (a) isolating cancer cells from a target region of the subject, wherein an alternating electric filed has been applied the target region of the subject prior to isolation of the cancer cells; (b) irradiating the isolated cancer cells to which the alternating electric field has been applied; (c) administering the isolated, irradiated cancer cells to the subject; and (d) applying an alternating electric field to the target region of the subject.

Illustrative embodiment 7. The method of any of illustrative embodiments 1-6a, wherein the cancer cells are selected from the group consisting of hepatocellular carcinoma cells, glioblastoma cells, pleural mesothelioma cells, differentiated thyroid cancer cells, advanced renal cell carcinoma cells, breast cancers, cervical cancers, ovarian cancers, pancreatic cancers, lung cancers, and combinations thereof.

Illustrative embodiment 8. The method of any of illustrative embodiments 1-7, wherein at least one of: the alternating electric field is applied at a frequency in a range of from about 50 kHz to about 1 MHz; the alternating electric field has a field strength of at least about 1 V/cm in at least a portion of the isolated cancer cells; and the period of time that the alternating electric field is applied is at least about 24 hours.

Illustrative embodiment 9. The method of any one of illustrative embodiments 1-8, wherein the cells are irradiated for a period of time sufficient to ensure that the cells are non-viable.

Illustrative embodiment 10. The method of illustrative embodiment 9, wherein the cells are irradiated for a period of time in a range of from about 24 hours to about 96 hours.

Illustrative embodiment 11. The method of any of illustrative embodiments 1-2a, wherein the immunogenic composition is formulated for intradermal, subcutaneous, intravenous, and/or intranodal administration.

Illustrative embodiment 12. The method of any of illustrative embodiments 1-2a or 11, further comprising the step of adding at least one additional composition to the immunogenic composition, wherein the at least one additional composition is selected from the group consisting of an adjuvant, a cytokine, an interferon, a TLR agonist, a STING (stimulator of interferon genes) agonist, GM-CSF, CD40L, Fms related tyrosine kinase 3 ligand (FLT3L), a C type Lectin Receptor (CLR), an anti-LAG3 agent, and combinations thereof.

Illustrative embodiment 13. The method of any one of illustrative embodiments 3-10, wherein the isolated, irradiated cancer cells are administered intradermally, subcutaneously, intravenously, and/or intranodally.

Illustrative embodiment 14. The method of any one of illustrative embodiments 3-10 and 13, wherein the isolated, irradiated cancer cells are administered to the subject in the form of at least one immunogenic composition, and wherein the at least one immunogenic composition further comprises at least one composition selected from the group consisting of an adjuvant, a cytokine, an interferon, a TLR agonist, a STING (stimulator of interferon genes) agonist, GM-CSF, CD40L, Fms related tyrosine kinase 3 ligand (FLT3L), a C type Lectin Receptor (CLR), an anti-LAG3 agent, and combinations thereof.

Illustrative embodiment 15. The method of any one of illustrative embodiments 1-14, further comprising the step sorting the non-viable, irradiated cancer cells to ensure that substantially no irradiated but still viable cancer cells are administered to the subject (i.e., present in the immunogenic composition).

Illustrative embodiment 16. An immunogenic composition prepared by the method of any of illustrative embodiments 1-2a, 7-12, and 15.

Illustrative embodiment 17. An immunogenic composition, comprising: a population of cancer cells isolated from a target region of a subject and treated ex vivo with an alternating electric field followed by irradiation.

Illustrative embodiment 18. An immunogenic composition, comprising: a population of cancer cells produced by treating a target region of a subject in vivo with an alternating electric field, isolating cancer cells from the treated target region of the subject, and irradiating the isolated, treated cancer cells.

Illustrative embodiment 19. The immunogenic composition of illustrative embodiment 17 or 18, wherein the cancer cells are selected from the group consisting of hepatocellular carcinoma cells, glioblastoma cells, pleural mesothelioma cells, differentiated thyroid cancer cells, advanced renal cell carcinoma cells, breast cancers, cervical cancers, ovarian cancers, pancreatic cancers, lung cancers, and combinations thereof.

Illustrative embodiment 20. The immunogenic composition of any of illustrative embodiments 17-19, wherein the immunogenic composition is formulated for intradermal, subcutaneous, intravenous, and/or intranodal administration.

Illustrative embodiment 21. The immunogenic composition of any one of claims 17-20, wherein the immunogenic composition further comprises at least one composition selected from the group consisting of an adjuvant, a cytokine, an interferon, a TLR agonist, a STING (stimulator of interferon genes) agonist, GM-CSF, CD40L, Fms related tyrosine kinase 3 ligand (FLT3L), a C type Lectin Receptor (CLR), an anti-LAG3 agent, and combinations thereof. Illustrative embodiment 22. The immunogenic composition of any one of illustrative embodiments 17-21, wherein the irradiated cancer cells are non-viable.

While the above disclosures describe the inventive concept(s) in conjunction with the specific experimentation, results, and language set forth, it is evident that many alternatives, modifications, and variations will be apparent to those skilled in the art. Accordingly, it is intended to embrace all such alternatives, modifications, and variations that fall within the spirit and broad scope of the present disclosure.

What is claimed is:

1. A method of preparing an immunogenic composition, the method comprising the steps of:
(1) isolating cancer cells from a target region of the subject;
(2) applying an alternating electric field to the isolated cancer cells; and
(3) irradiating the isolated cancer cells to which the alternating electric field has been applied to provide the immunogenic composition.

2. The method of claim 1, wherein in step (1), the cancer cells are selected from the group consisting of hepatocellular carcinoma cells, glioblastoma cells, pleural mesothelioma cells, differentiated thyroid cancer cells, advanced renal cell carcinoma cells, breast cancers, cervical cancers, ovarian cancers, pancreatic cancers, lung cancers, and combinations thereof.

3. The method of claim 1, wherein in step (2), at least one of:
the alternating electric field is applied at a frequency in a range of from about 50 kHz to about 1 MHz;
the alternating electric field has a field strength of at least about 1 V/cm in at least a portion of the isolated cancer cells; and
the period of time that the alternating electric field is applied is at least about 24 hours.

4. The method of claim 1, wherein in step (3), the cells are irradiated for a period of time sufficient to ensure that the cells are non-viable.

5. The method of claim 1, further comprising the step sorting the non-viable, irradiated cancer cells to ensure that substantially no irradiated but still viable cancer cells are present in the immunogenic composition.

6. The method of claim 1, wherein the immunogenic composition is formulated for intradermal, subcutaneous, intravenous, and/or intranodal administration.

7. A method of treating cancer in a subject, the method comprising the steps of:
(1) isolating cancer cells from a target region of the subject;
(2) applying an alternating electric field to the isolated cancer cells;
(3) irradiating the isolated cancer cells to which the alternating electric field has been applied;
(4) administering the isolated, irradiated cancer cells to the subject; and
(5) applying an alternating electric field to the target region of the subject.

8. The method of claim 7, wherein in step (1), the cancer cells are selected from the group consisting of hepatocellular carcinoma cells, glioblastoma cells, pleural mesothelioma cells, differentiated thyroid cancer cells, advanced renal cell carcinoma cells, breast cancers, cervical cancers, ovarian cancers, pancreatic cancers, lung cancers, and combinations thereof.

9. The method of claim 7, wherein in step (2), at least one of:
the alternating electric field is applied at a frequency in a range of from about 50 kHz to about 1 MHz;
the alternating electric field has a field strength of at least about 1 V/cm in at least a portion of the isolated cancer cells; and
the period of time that the alternating electric field is applied is at least about 24 hours.

10. The method of claim 7, wherein in step (3), the cells are irradiated for a period of time sufficient to ensure that the cells are non-viable.

11. The method of claim 10, wherein the cells are irradiated for a period of time in a range of from about 24 hours to about 96 hours.

12. The method of claim 7, wherein in step (4), the isolated, irradiated cancer cells are administered intradermally, subcutaneously, intravenously, and/or intranodally.

13. The method of claim 7, wherein in step (4), the isolated, irradiated cancer cells are administered to the subject in the form of at least one immunogenic composition, and wherein the at least one immunogenic composition further comprises at least one composition selected from the group consisting of an adjuvant, a cytokine, an interferon, a TLR agonist, a STING (stimulator of interferon genes) agonist, GM-CSF, CD40L, Fms related tyrosine kinase 3 ligand (FLT3L), a C type Lectin Receptor (CLR), an anti-LAG3 agent, and combinations thereof.

14. The method of claim 7, wherein in step (5), at least one of:
the alternating electric field is applied at a frequency in a range of from about 50 kHz to about 1 MHz;
the alternating electric field has a field strength of at least about 1 V/cm in at least a portion of the target region of the subject; and
the period of time that the alternating electric field is applied is at least about 24 hours.

15. The method of claim 7, further defined as a method of reducing a volume of a tumor and/or preventing an increase of volume of the tumor, wherein the tumor is present in a body of a living subject and includes a plurality of cancer cells.

16. An immunogenic composition, comprising:
a population of cancer cells isolated from a target region of a subject and treated ex vivo with an alternating electric field followed by irradiation.

17. The immunogenic composition of claim 16, wherein the cancer cells are selected from the group consisting of hepatocellular carcinoma cells, glioblastoma cells, pleural mesothelioma cells, differentiated thyroid cancer cells, advanced renal cell carcinoma cells, breast cancers, cervical cancers, ovarian cancers, pancreatic cancers, lung cancers, and combinations thereof.

18. The immunogenic composition of claim 16, wherein the immunogenic composition is formulated for intradermal, subcutaneous, intravenous, and/or intranodal administration.

19. The immunogenic composition of claim 16, wherein the immunogenic composition further comprises at least one composition selected from the group consisting of an adjuvant, a cytokine, an interferon, a TLR agonist, a STING (stimulator of interferon genes) agonist, GM-CSF, CD40L, Fms related tyrosine kinase 3 ligand (FLT3L), a C type Lectin Receptor (CLR), an anti-LAG3 agent, and combinations thereof.

20. The immunogenic composition of claim 16, wherein the irradiated cancer cells are non-viable.
